(19) United States
(12) Patent Application Publication
(10) Pub. No.: US 2025/0090469 A1
(43) Pub. Date: Mar. 20, 2025

(54) PRODUCT CONTAINING PLANT DERIVED EXTRACELULAR VESICLES TO HELP SLIMMING AND REDUCES THE APPEARANCE OF CELLULITE

(71) Applicants: Merve YILDIRIM CANPOLAT, Istanbul (TR); Polen KOCAK DENIZCI, Istanbul (TR); CELLESTETIX GENETIK DANISMANLIK ARGE VE BIYOTEKNOLOJI ANONIM SIRKETI, Istanbul (TR)

(72) Inventors: Merve YILDIRIM CANPOLAT, Istanbul (TR); Polen KOCAK DENIZCI, Istanbul (TR)

(73) Assignees: Merve YILDIRIM CANPOLAT, Istanbul (TR); Polen KOCAK DENIZCI, Istanbul (TR); CELLESTETIX GENETIK DANISMANLIK ARGE VE BIYOTEKNOLOJI ANONIM SIRKETI, Istanbul (TR)

(21) Appl. No.: 18/290,811

(22) PCT Filed: Dec. 20, 2022
(86) PCT No.: PCT/TR2022/051530

§ 371 Date: Jan. 22, 2024

(30) Foreign Application Priority Data
Jan. 21, 2022 (TR) 2022/000785

(51) Int. Cl. A61K 9/50; A61K 36/88

(52) U.S. Cl. CPC A61K 9/5068; A61K 36/88

(57) ABSTRACT

A product containing a plant exosome is provided, where the product is used in the treatment of obesity, overweight and fat follicles reduction. With the product, a low-cost product is obtained that does not cause toxic effects in the human body, enables the cells to transform into the old stem cell form by reducing the fat follicles, does not cause damage to healthy cells, does not require any surgical procedure, does not create infection risk. Pineapple can be used as a herbal source in the product.

### CROSS REFERENCE TO THE RELATED APPLICATIONS

This application is the national phase entry of International Application No. PCT/TR2022/051530, filed on Dec. 20, 2022, which is based upon and claims priority to Turkish Patent Application No. 2022/000785, filed on Jan. 21, 2022, the entire contents of which are incorporated herein by reference.

### TECHNICAL FIELD

The invention relates to a product containing plant derived extracellular vesicle complex, which helps to weaken by shrinking the fat follicles without having a toxic effect on the viability of the fat-derived stem cells.

### BACKGROUD

WHO (WHO), together with the National and International medical and scientific communities, defined obesity as a chronic progressive, non-communicable epidemic disease caused by many environmental and genetic factors. Obesity is a serious disease that threatens individuals and public health and is rapidly increasing worldwide. Due to its ever-increasing nature, obesity requires a lifelong struggle and control. According to WHO data, there are 500 million obese adults in the world and 65% of the world's population lives in countries where obesity is a problem, and obesity-related mortality rates are very high in these countries. Recent studies have found that 250 million children worldwide have obesity. Over the past 20-25 years, the prevalence of obesity has been increasing at an alarming rate. Sleep apnea and other respiratory problems due to weight gain can trigger appetite by disrupting sleep quality and duration. Weight gain also causes other diseases such as hypertension, diabetes, heart disease, osteoarthritis and depression.

The high amount of vitamin C contained in pineapple has an important role in antioxidant-based treatments to prevent complications caused by fat accumulation in obesity. Phenolic compounds in pineapple contribute to the treatment of obesity. According to the literature, it is found that pineapple juice suppresses obesity caused by high-calorie diet by reducing lipids in the body, weight gain and hepatic lipid accumulation. These effects have been shown to regulate fat metabolism in the body by down-regulating lipogenesis and up-regulating fatty acid oxidation of genes in lipid metabolism.

These findings show that pineapple juice has no obvious harmful effects and may be an effective agent against obesity (Samir et al. 2018). In intercellular communication, extracellular vesicles take part in long-distance communication and carry cell-specific substances such as DNA, RNA and proteins. The fact that exosomes contain some important biomarkers used in the diagnosis of diseases has led to the idea of using them in the diagnosis of diseases and has led to an increased interest in exosomes (İnanir et al. 2020).

As a result of preclinical studies in the laboratory that we have done so far, the effect of pineapple juice and pineapple-derived nanoparticles on fat follicles was compared and it was observed that nanoparticles obtained from pineapple were more effective in decreasing the amount of fat follicles. According to the results, it is predicted that it can be used as an aid to weight loss due to the reduction of the fat follicles. It is aimed to develop an affordable, herbal product.

Studies have shown that extracellular vesicles released by plants such as garlic, grapes, grapefruit, ginger, and carrots have anti-inflammatory and anti-cancer effects. However, the effect of pineapple plant-derived extracellular vesicles on the basic processes of weight loss or obesity is still an important subject of research.

### SUMMARY

This invention was developed as a weight loss aid technology strategy containing pineapple derived extracellular vesicles. In addition, the fact that extracellular vesicle-derived products are cell-free, and it has revealed that cellular therapy can prevent the problems that may arise. Exosomes are ready-to-use therapeutics. It can be stored at low temperatures and safely delivered to patients without the need for toxic cryopreservative agents and ultra-freezers.

Based on this information, it is aimed to use this technology in alternative medicine as a weight loss-supporting product obtained from Ananas Comosus, Pineapple-derived plant exosomes complexes.

First, exosomes were isolated from the pineapple plant with the known two-phase method and characterization experiments were completed to prove that the nanoparticle we obtained was an exosome. One of these methods is to measure the size and charge of exosomes in a zetasizer and to show their size and shape in the electron microscope. In addition, the special surface markers on it were examined by flow cytometry method. Cell viability was demonstrated by MTS, apoptosis and annexin staining tests, as different concentrations of isolated exosomes did not have a cytotoxic effect on adipose stem cells in vitro.

The fat reduction effect of exosomes has been demonstrated by visualizing the fat follicles formed inside the cell with Oil Red staining. In the next step, to make observations at the gene level, the expression of PPAR gamma, adiponectin, LPL, aP2 genes were examined by using the Polymer Chain Reaction (RT PCR) method with RNA isolation from cells given the optimal dose of exosomes. One of the main adipogenic markers, PPAR gamma, has been shown in studies to increase FGF21 with its increased activation in mice and monkeys, and this increased FGF21 reduces blood sugar, cholesterol, and body weight without causing any cancer or side effects. Since the increased energy consumption of F GF21 increases with the activation of PPAR gamma, it plays a role in the consumption and excretion of fats in the body (Gregor et al, 2011). In the literature, the LPL enzyme is secreted by fat cells to form fat stores, and it is said that this LPL activity increases during weight gain (Wang et al. 2009). When adiponectin levels in the blood taken from obese individuals were examined, it was observed that adiponectin levels were lower than those of normal weight, and a decrease in adiponectin levels was a positive factor (Wade et al. 2009). In line with the results we have obtained, a Biotechnology product containing pineapple exosome which helps weight loss has been developed.

### BRIEF DESCRIPTION AND OBJECTS OF THE INVENTION

The aim of the invention is to obtain an effective plant exosome-derived product that can be used in the treatment of obesity or as an aid to weight loss.

Another aim of the invention is; to obtain a low-cost product.

Another aim of the invention is; to obtain a natural product that does not experience chemical contamination in the body caused by synthetic drugs.

Another aim of the invention is; It is to provide a practical and easily accessible product as the plants to be used in the treatment grow easily and a large scale exosome is obtained in a short time.

### BRIEF DESCRIPTION OF THE DRAWINGS

Figures related to “A Product Containing Plant Exosome” developed to achieve the purpose of the invention are shown;

FIGS. 1A-1D: Characterization of plant exosome from pineapple plant, respectively FIG. 1A) Scanning electron microscopy (SEM) to monitor the morphology of exosomes; FIG. 1B) Flowcytometry plot of pineapple exosomes using surface markers CD63, CD9 and HSP70; FIG. 1C) Analysis of exosome size by Zeta potential; and FIG. 1D) Demonstrated by nanoparticle tracking analysis (NTA).

FIG. 2: Cell viability analysis of HADSc human adipose-derived stem cells at 24, 48 and 72 hours after treatment with pineapple exosome applied at a specific concentration (100 μg/mL).

FIG. 3: HADSc human adipose-derived stem cells were differentiated in culture medium for 14days in vitro under adipogenic induction and exosome treatment was applied for 11 days post-differentiation. Fluorescent microscope view of the control group and the group treated with 100 μg/ml pineapple exosome at the time of completion of adipogenic differentiation and after 11 days of exosome treatment to evaluate the reduction of fat follicles in vitro.

FIG. 4A: PPAR ELISA analysis of HADSc human adipose-derived stem cells from media collected every other day of 11-day treatment of 100 μg/ml pineapple exosomes.

FIG. 4B: LPL ELISA analysis of HADSc human adipose-derived stem cells from media collected every other day of 11-day treatment of 100 μg/ml pineapple exosomes.

FIGS. 5A-5D: HADSc human adipose-derived stem cells were cultured in vitro for 14 days using the StemPro Adipogenesis Differentiation Kit, and after adipogenic differentiation, the cells were treated for 11 days with a pineapple exosome concentration of 100 μg/ml.

Quantitative RT-PCR analysis of PPAR gamma, adiponectin, LPL and aP2 expression levels of human adipose-derived stem cells (HA Sc) is shown.

### DETAILED DESCRIPTION OF THE EMBODIMENTS

### 1. Experimental Study

### Preparation of Plant Originated Exosome

Plant-derived exosomes from pineapple, lemon, tomato, wheat can be used alone or in combinations as a plant source in the plant exosome-containing product of the invention. Pineapple was chosen preferably in experimental studies. Pineapples were juiced for exosome isolation.

Pineapple juice was centrifuged at 1000 xg for 10 minutes, 3200 xg for 20 minutes, and 15000 xg for 60 minutes, and pineapple juice-derived exosomes were isolated using two phase polymer systems. Isolated exosomes were dissolved in 0.9% isotonic serum.

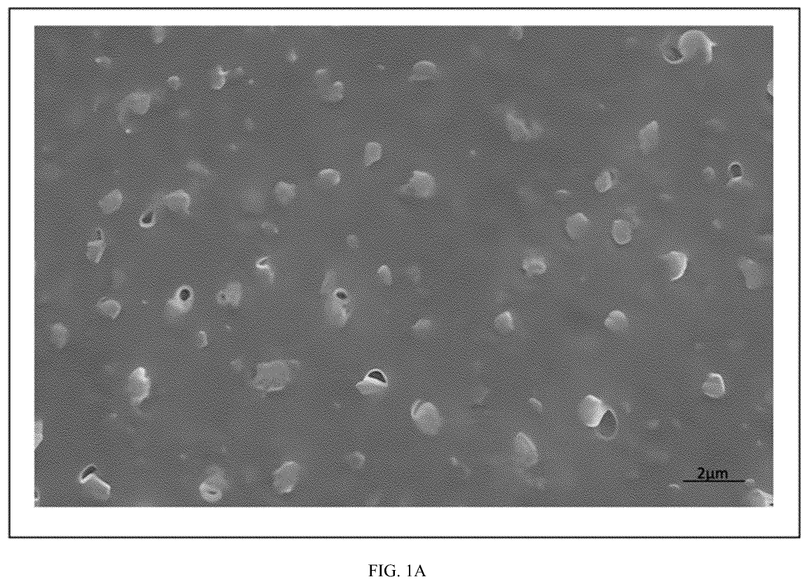

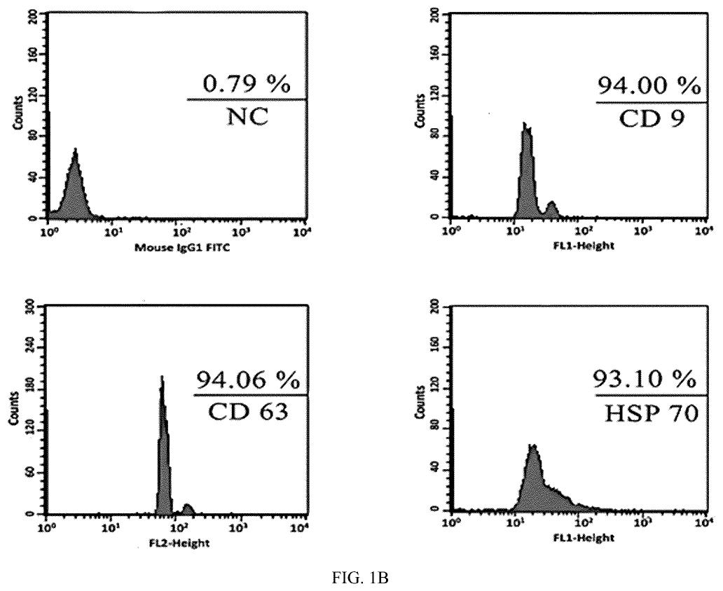

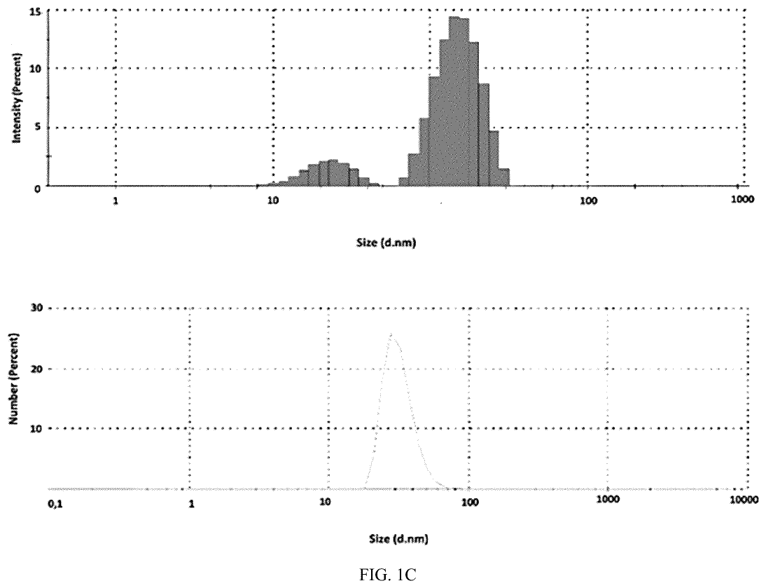

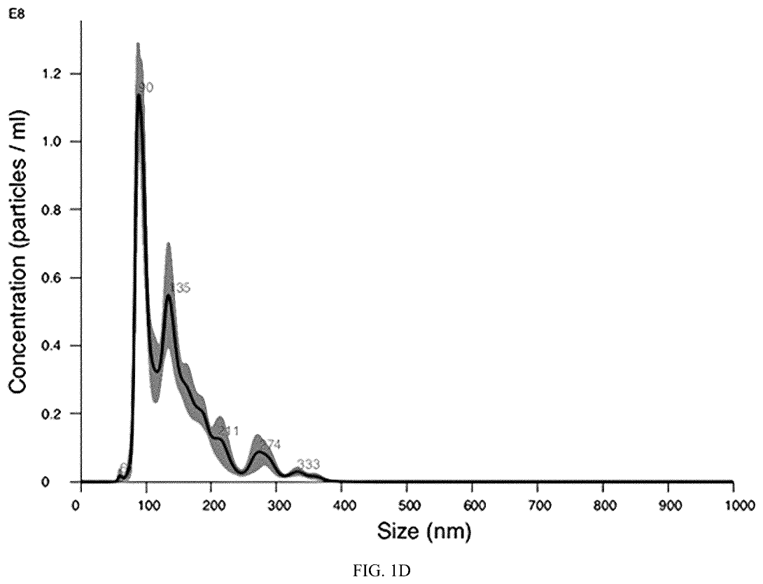

The product exosomes of the invention, which are isolated and in serum form, were observed using scanning electron microscopy (FIGS. 1A-1D). Afterwards, the presence of exosomes was observed in flow cytometry by incubating with the HSP 70 marker. (FIGS. 1A-1D).

### 2. Development of the Treatment Strategy

### Determination of Cell Toxicity

The toxic effect of the prepared product, which is the subject of the invention, was determined using the MTS method in the literature (Yalvaç et al, 2009).

Chemical molecules were applied on human adipose stem cells (HA Sc), which were prepared in the medium at the indicated concentrations and counted in 96-well culture dishes (5000 cells/well). The toxicity response of the cells to the chemicals was determined by measuring the cell viability for 3 days. Cell viability analysis was performed according to the MTS method.

MTS is a colorimetric method used to measure mitochondrial enzyme activity. The effect of the applied substance on the viability was investigated within the specified time interval by applying different doses to the cells previously seeded in the wells. The MTS agent, which is mixed with the media, turns dark with the increase in the number of viable cells. The resulting color change was evaluated as an absorbance measurement using an ELISA reader. The product developed within the scope of the invention has been prepared in such a way that results can be obtained at 24, 48 and 72 hours in healthy cells with concentrations of 30 μg/ml, 50 μg/ml, 75 μg/ml, 100 μg/ml and 200 μg/ml.

### Quantitative RT-PCR Analysis

The rate of adipogenic differentiation in human adipose-derived stem cells was investigated by isolation of RNAs and differences in gene expressions. These hADSCs were seeded in 6-well plate and 100,000 cells were seeded in each well. First, each well was cultured for 14 days in vitro using the StemPro Adipogenesis Differentiation Kit. After adipogenic differentiation, two wells in the 6-well plate were treated with pineapple exosome at a concentration of 100 μg/ml for 11 days, and cells were refreshed with fresh media-exosome solutions every other day. The other two wells of the plate were treated only with DMEM low glucose medium (10% FBS and 5% PSA) for 11 days. Macherey-Nagel's NucleoSpin RNA Isolation Kit was used for total RNA isolation, and BIORAD's Script cDNA Synthesis Kit was used to obtain cDNA after RNA isolation. Expression of adiponectin, LPL, PPAR-gamma and aP2 genes were measured using RealTime-PCR.

### Oil Red Staining

In this study, hADSCs were cultured for 14 days in vitro using the StemPro Adipogenesis Differentiation Kit to induce adipogenic differentiation, and after completion of adipogenic differentiation, cells were cultured for 11 days with DMEM (10% FBS, 5% PSA) and 100 μg/ml pineapple exosome. Oil droplets in fat cells were examined by Oil-Red staining in FIG. 3. As seen in FIG. 3, control cells were incubated with DMEM only and normal fat formation and morphology were seen in adipo cells. Looking at FIG. 3, the 100 μg/ml concentration of pineapple exosome decreased the formation of oil follicles in fat cells.

### 3. Experimental Results

With the developed isolation method, exosomes were obtained preferably using pineapple. This product has been subjected to characterization experiments and its effect on the reduction of fat follicles in fat cells has been proven by the results. It was found that the data obtained in the electron microscope image and the visible vesicles were similar to the exosomes found in the literature. (FIGS. 1A-1D) Flow cytometry results show that exosomes express at least 79.91% of the surface marker. This is one of the proofs, based on MISEV2020 Standards, that the molecule we obtained is an exosome (FIGS. 1A-1D).

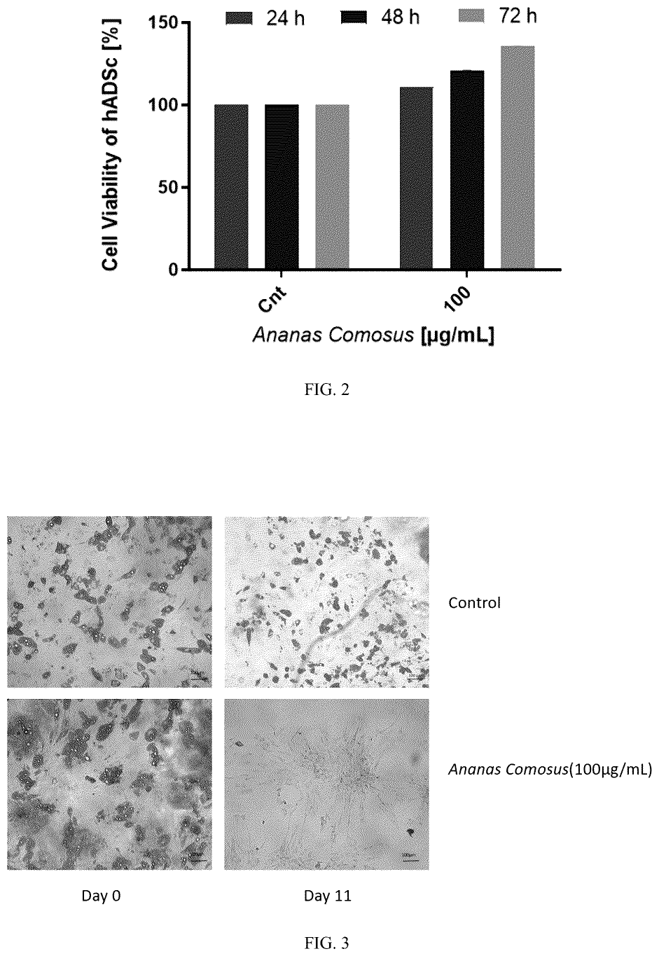

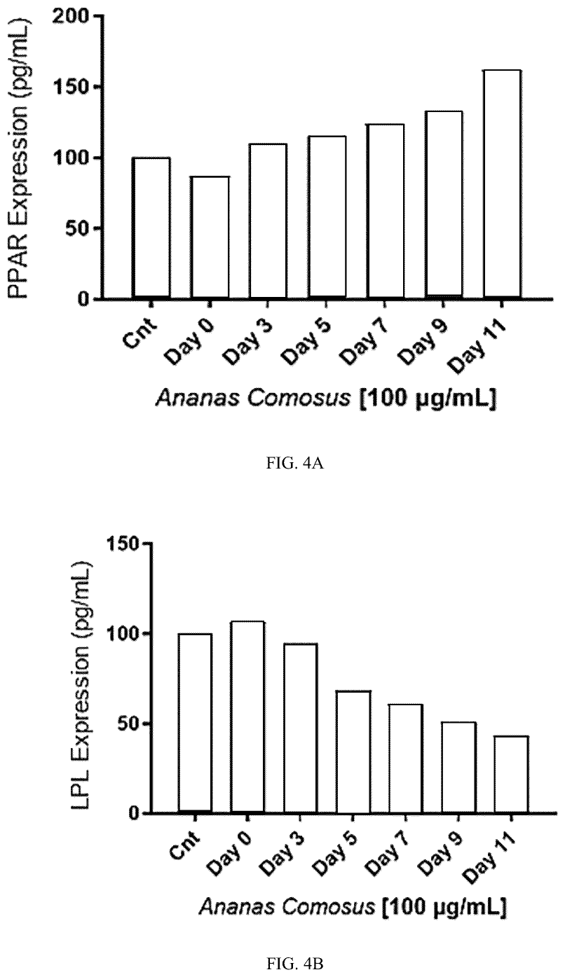

It has been proven by the MTS result that when healthy cells are exposed to plant exosome, it causes proliferation in cells. The amount of absorbance increased compared to the control indicates increased viability. Analyzes made in this direction have proven that the pineapple exosome increases the rate of division in healthy cells (FIG. 2).

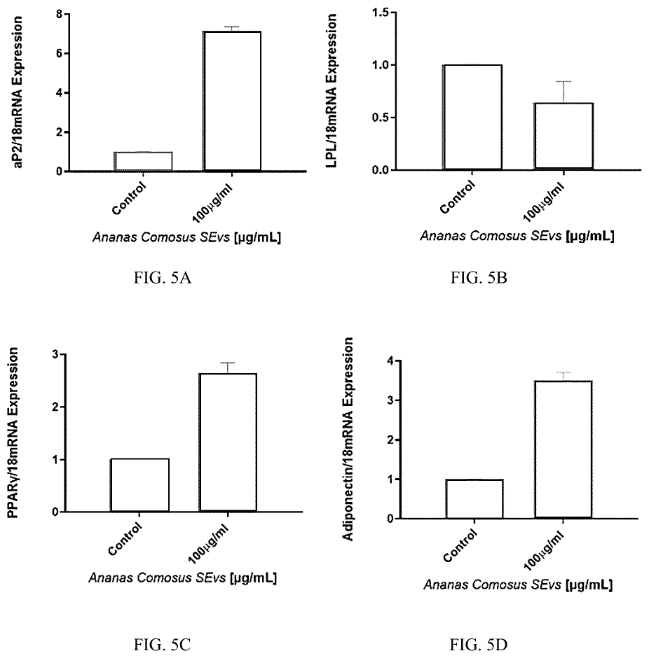

The effect of exosomes on cells treated with exosomes after adipogenic differentiation was demonstrated by Real-Time PCR and Oil-Red staining method. In adipogenic differentiation, oils formed in hADSCs were observed by Oil-Red staining (FIG. 3 and FIGS. 5A-5D). This staining is a method used to identify hydrophobic lipids present in an aqueous medium. FIGS. 5A-5D show the expression levels of PPAR, aP2, adiponectin and LPL in the pineapple exosome. Looking at these

When we research the literature, while the levels of PPAR, aP2 and adiponectin increase, on the contrary, the level of LPL expression should decrease. Looking at FIGS. 5A-5D, it is seen that the results are in this direction.

In line with these results, it is seen that the plant exosome does not have toxic effect on human adipose-derived stem cells (HA Sc), whereas accelerates the conversion of fat follicles to the old stem cell form and increases their yield after the adipogenic differentiation is completed in these cells.

As a result of the experimental results; It has been proven that the product of the invention triggers the reduction of fat cells in the body by decreasing fat follicles. As a result of the experimental model created based on the oil red staining and PCR results, it has been proven that the fat follicles in the cells treated with the product of the invention reduce the fat follicles much faster than the untreated control cells. This showed that the obtained product, especially from pineapple exosome, positively affects and accelerates the process in the reduction of fat follicles and in the models of returning to the stem cell, which is the old form of the cells.

### 4. Application of the Invention

The invention is applied in stem cells of human adipose origin. The product subject to the invention can be produced primarily in liquid form, in serum form, or as a solid. These can be in the form of serum, syrup, tablet, medicine, gel, cream. At the same time, it can be used as a nano carrier molecule, allowing another active substance (such as medicine, chemical) to be transported into the cell. This method of transport can also be used in different forms and formulations as mentioned above.

### REFERENCES

[1] Sun, K., Kusminski, C. M., & Scherer, P. E. (2011). Adipose tissue remodeling and obesity. Journal of Clinical Investigation.
[2] James, A. W. (2013). Review of Signaling Pathways Governing MSC Osteogenic and Adipogenic Differentiation. Scientifica.
[3] Scott, M. A., Nguyen, V. T., Levi, B., & James, A. W. (2011). Current Methods of Adipogenic Differentiation of Mesenchymal Stem Cells. Stem Cells and Development.
[4] Cristancho, A. G., & Lazar, M. A. (2011). Forming functional fat: A growing understanding of adipocyte differentiation. Nature Reviews Molecular Cell Biology.
[5] Raposo, G., & Stoorvogel, W. (2013). Extracellular vesicles: Exosomes, microvesicles, and friends. Journal of Cell Biology.
[6] Théry, C., Zitvogel, L., & Amigorena, S. (2002). Exosomes: Composition, biogenesis and function. Nature Reviews Immunology.
[7] Zhang, J., Li, S., Li, L., Li, M., Guo, C., Yao, J., & Mi, S. (2015). Exosome and exosomal microRNA: Trafficking, sorting, and function. Genomics, Proteomics and Bioinformatics.
[8] Colombo, M., Raposo, G., & Théry, C. (2014). Biogenesis, Secretion, and Intercellular Interactions of Exosomes and Other Extracellular Vesicles. Annual Review of Cell and Developmental Biology.
[9] Valadi, H., Ekström, K., Bossios, A., Sjöstrand, M., Lee, J. J., & Lötvall, J. O. (2007). Exosome-mediated transfer of mRNAs and microRNAs is a novel mechanism of genetic exchange between cells. Nature Cell Biology.
[10] Gregor, M. F., & Hotamisligil, G. S. (2011). Inflammatory Mechanisms in Obesity. Annual Review of Immunology.
[11] Vidal-Puig, A., Jimenez-Liñan, M., Lowell, B. B., Hamann, A., Hu, E., Spiegelman, B., . . . Moller, D. E. (1996). Regulation of PPAR γ gene expression by nutrition and obesity in rodents. Journal of Clinical Investigation.
[12] Wang, H., & Eckel, R. H. (2009). Lipoprotein lipase: from gene to obesity. American Journal of Physiology-Endocrinology and Metabolism.
[13] Mead, J. R., Irvine, S. A., & Ramji, D. P. (2002). Lipoprotein lipase: Structure, function, regulation, and role in disease. Journal of Molecular Medicine.
[14] Wade, T. E., Mathur, A., Lu, D., Swartz-Basile, D. A., Pitt, H. A., & Zyromski, N. J. (2009). Adiponektin Receptor-1 Expression Is Decreased in the Pancreas of Obese Mice. Journal of Surgical Research.

What is claimed is:

1. A product for a treatment of fat cells, comprising a plant exosome.

2. The product according to claim 1, wherein a source of the plant exosome is a pineapple.

3. The product according to claim 2, wherein the source of the plant exosome is selected from one or a combination of the pineapple, a tomato, wheat, and a lemon.

4. The product according to claim 1, wherein the product is allowed to be configured alone or in combination with the plant exosome.

5. The product according to claim 1, wherein the product is configured to protect cell viability.

6. The product according to claim 1, wherein the product is configured as a cell division enhancer.

7. The product according to claim 1, wherein the product does not have toxic effects on healthy cells in a body.

8. The product according to claim 1, wherein the product is in a form of serum, syrup, tablet, medicine, gel, or cream.

9. The product according to claim 1, wherein the product is effective in human adipose-derived stem cells.

10. The product according to claim 2, wherein the product is configured to protect cell viability.

11. The product according to claim 2, wherein the product is configured as a cell division enhancer.

12. The product according to claim 3, wherein the product is configured as a cell division enhancer.

13. The product according to claim 2, wherein the product does not have toxic effects on healthy cells in a body.

14. The product according to claim 3, wherein the product does not have toxic effects on healthy cells in a body.

15. The product according to claim 4, wherein the product does not have toxic effects on healthy cells in a body.

16. The product according to claim 2, wherein the product is in a form of serum, syrup, tablet, medicine, gel, or cream.

17. The product according to claim 3, wherein the product is in a form of serum, syrup, tablet, medicine, gel, or cream.

18. The product according to claim 4, wherein the product is in a form of serum, syrup, tablet, medicine, gel, or cream.

19. The product according to claim 5, wherein the product is in a form of serum, syrup, tablet, medicine, gel, or cream.

20. The product according to claim 2, wherein the product is effective in human adipose-derived stem cells.
